(19) United States
(12) Patent Application Publication
(10) Pub. No.: US 2003/0125788 A1
(43) Pub. Date: Jul. 3, 2003

(54) Self-propelled, intraluminal device with electrode configuration and method of use

(76) Inventor: Gary L. Long, Gerards Cross (GB)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/281,586

(22) Filed: Oct. 28, 2002

Related U.S. Application Data
(60) Provisional application No. 60/344,426, filed on Nov. 9, 2001.
(60) Provisional application No. 60/344,429, filed on Nov. 9, 2001.

(51) Int. Cl. A61N 1/00

(52) U.S. Cl. 607/133

(57) ABSTRACT

A medical device is provided comprising a capsule for introduction into a bodily lumen. The capsule can include a balloon filled with a conductive fluid, or a mechanism for actuating wings supporting electrodes. An umbilicus can attach to the trailing end of the capsule. A control unit controls propulsion of the capsule through the bodily lumen.

This application claims priority to the following provisional patent applications: “Method for Providing Access to Luminal Tissue”, Serial No. 60/344,426, filed Nov. 9, 2001 in the name of Long et al.; and “Luminal Propulsive Device Having a Generally Continuous Passageway”, Serial No. 60/344,429, filed Nov. 9, 2001 in the name of Long et al.

### FIELD OF THE INVENTION

The present invention relates to a medical device that self-propels within a lumen of a patient's body.

### BACKGROUND

A physician typically accesses and visualizes tissue within a patient's gastrointestinal (GI) tract with a long, flexible endoscope. For the upper GI, a physician may insert a gastroscope into the sedated patient's mouth to examine and treat tissue in the esophagus, stomach, and proximal duodenum. For the lower GI, a physician may insert a colonoscope through the sedated patient's anus to examine the rectum and colon. Some endoscopes have a working channel, typically about 2.5-3.5 mm in diameter, extending from a port in the handpiece to the distal tip of the flexible shaft. A physician may insert medical instruments into the working channel to help diagnose or treat tissues within the patient. Physicians commonly take tissue biopsies from the mucosal lining of the GI tract using a flexible, biopsy forceps through the working channel of the endoscope.

Insertion of a flexible endoscope, especially into the colon, is usually a very time-consuming and uncomfortable procedure for the patient, even when sedated with drugs. A physician often needs several minutes to push a flexible endoscope through the convoluted sigmoid, descending, transverse, and ascending portions of the colon. The physician may diagnose and/or treat tissues within the colon either during insertion or removal of the endoscope. Often the flexible endoscope “loops” within the colon, such as at the sigmoid colon or at the splenic flexure of the colon, so that the inserted length of the endoscope is longer than the portion of colon containing it. Depending on the anatomy of the patient and the skill of the physician in manipulating the flexible endoscope, some portions of the colon may be unexamined, thus increasing the risk of undiagnosed disease.

Given® Engineering LTD, Yoqneam, Israel, sells a device in the U.S. called the M2A™ Swallowable Imaging Capsule. The device contains a tiny video camera, battery, and transmitter. It is propelled through the gastrointestinal tract by natural peristalsis. The device is currently used for diagnostic purposes and passes through the intestinal tract with a velocity determined by the natural, peristaltic action of the patient’s body. World Publication No. WO 0108548A1 filed by C. Mosse, et al. describes a self-propelling device adapted to travel through a passage having walls containing contractile tissue. The applicants disclose that the device is particularly useful as an enteroscope and may also carry objects such as feeding tubes, guide wires, physiological sensors or conventional endoscopes within the gut. A summary of other alternatives to push endoscopy can be found in “Technical Advances and Experimental Devices for Enteroscopy” by C. Mosse, et al, published in Gastrointestinal Endoscopy Clinics of North America, Volume 9, Number 1, January 1999: pp. 145-161.

Since it is desirable to minimize the electrical power dispersed into a patient's body during a medical treatment, features may be provided for improving electrical conduction between the electrodes of a self-propelled, intraluminal device and the contractile tissue. Many electrode geometries are well known in the art, including electrodes for electrosurgical devices. In general for electrically stimulating contractile tissue to contract, it is desirable to achieve a broad area of intimate contact between the electrodes and the contractile tissue. It would also be desirable to include means to disperse electrical energy uniformly to the contractile tissue around the circumferential portion of the luminal wall that acts upon and propels the device. What is needed, therefore, is a self-propelled, intraluminal device that includes means for improved stimulation of the contractile tissue of the luminal wall, to increase the speed and force of self-propulsion.

### SUMMARY OF THE INVENTION

In one embodiment, the present invention provides a medical device comprising a capsule adapted for travel in a body lumen. The capsule comprises a movable portion, the movable portion movable from a contracted configuration and an expanded configuration, and a lumen tissue stimulator associated with the movable portion of the capsule. The lumen stimulator can comprise one or more electrodes, and the movable portion can comprise one or more wings. The wings can each support an electrode and be biased to contracted configuration. An actuator can be used to extend the wings and the electrodes to an expanded configuration, to position the electrodes in a desired position with respect to lumen tissue. The actuator can be an inflatable member, such as a balloon, or a mechanical mechanism, such as a cable or linkage assembly. In one embodiment, an inflatable member is inflated by pressurizing a conductive fluid within the inflatable member.

In another embodiment, the present invention provides a method for accessing tissue within a body lumen. The method can comprise the steps of providing a capsule, the capsule supporting at least one electrode; and actuating a portion of the capsule to move the electrode into a desired position with respect to tissue within the lumen. The device can comprise a working channel, and the method can comprise accessing tissue through the device from a point outside the patient's body.

### BRIEF DESCRIPTION OF THE DRAWINGS

We have set forth the novel features of the invention with particularity in the appended claims. To fully understand the invention, however, please refer to the following description and accompanying drawings.

FIG. 1 is a cross sectional view of a wall 14 of a hollow organ such as the colon.

FIG. 2 shows a medical device 101, which includes a capsule 100, an umbilicus 140, a control unit 20, a DC power source 60, a display 80, and a fluid system 40.

FIG. 3 is a side view of capsule 100 of medical device 101 shown in FIG. 2, with a portion of a balloon 108 removed to reveal a first electrode 110.

FIG. 4 is an end view of capsule 100 shown in FIG. 2.

FIG. 5 is a sectional view of capsule 100 shown in FIG. 2, showing a fluid 122 inside of balloon 108, and a leading end 104 that contains a visualization device 112 and a lighting device 114.

FIG. 6 is a side view of a capsule 200 of a medical device 200, wherein capsule 200 includes a working channel 217.

FIG. 7 is an end view of capsule 200 of FIG. 6.

FIG. 8 is a sectional view of capsule 200 of FIG. 6, showing a fluid 222 contained in a balloon 208, and a visualization device 212 positioned inside of working channel 217.

FIG. 9 shows capsule 100 of FIG. 2 inside of a bodily lumen 15 while wall 14 is relaxed.

FIG. 10 shows capsule 100 of FIG. 2 self-propelling in a forward (right) direction inside of bodily lumen 15, while capsule 100 capacitively stimulates wall 14.

FIG. 11 is a distal end view of a capsule 300 of a medical device 301, which includes a first electrode 331, a second electrode 333, a third electrode 335, and a fourth electrode 337, shown in a normally contracted position.

FIG. 12 shows medical device 301, which includes capsule 300, a control unit 22, and an inflator 52.

FIG. 13 is a proximal end view of capsule 300 of FIG. 12.

FIG. 14 is a sectional view of capsule 300, which includes a working channel 317 and a balloon 308.

FIG. 15 is a distal end view of a capsule 400, which includes a first electrode 431, a second electrode 433, a third electrode 435, and a fourth electrode 437, shown in a normally expanded position.

FIG. 16 is a proximal end view of capsule 400 of FIG. 16.

FIG. 17 shows a medical device 401, which includes capsule 400 of FIG. 2, an umbilicus 440, a cable actuator 27, a control unit 24, a DC power source 25, and a display 26.

FIG. 18 shows capsule 400 in bodily lumen 15, while capsule 400 is in an expanded configuration and wall 14 of the hollow organ is relaxed.

FIG. 19 shows capsule 400 in bodily lumen 15, while capsule 400 is electrically stimulating wall 14, causing wall 14 to force capsule 14 in a contracted configuration and propelling capsule 400 in a forward (right) direction.

### DETAILED DESCRIPTION OF THE INVENTION

The present invention comprises a self-propelled intraluminal medical device. By way of example, the present invention is illustrated and described for application in the colon of a human patient. However, the present invention is applicable for use in the bodily lumens of other hollow organs in humans and in other mammals.

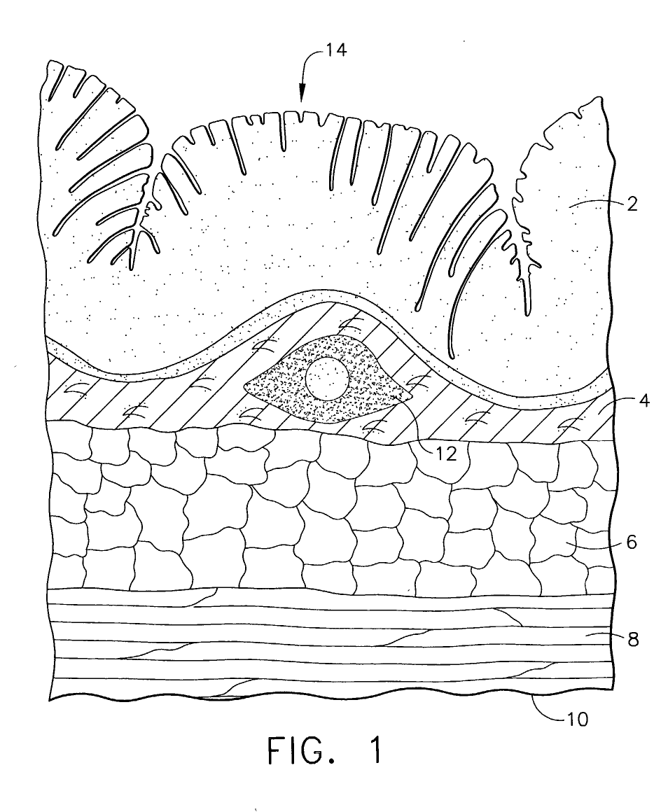

FIG. 1 shows a section of a wall 14 of the mammalian colon, and includes a mucosal layer 2, a submucosal layer 4 (shown with a lymph node 12), a circular muscular layer 6, a longitudinal muscular layer 8, and a serosa 10. Natural peristalsis is a progressive wavelike contraction of wall 14 that occurs involuntarily and is normally stimulated by distention of wall 14 from the contents within. Circular muscular layer 6 and longitudinal muscular layer 8 comprise the contractile tissue and contract when electrically stimulated, causing an instantaneous circumferential reduction of that portion of the lumen.

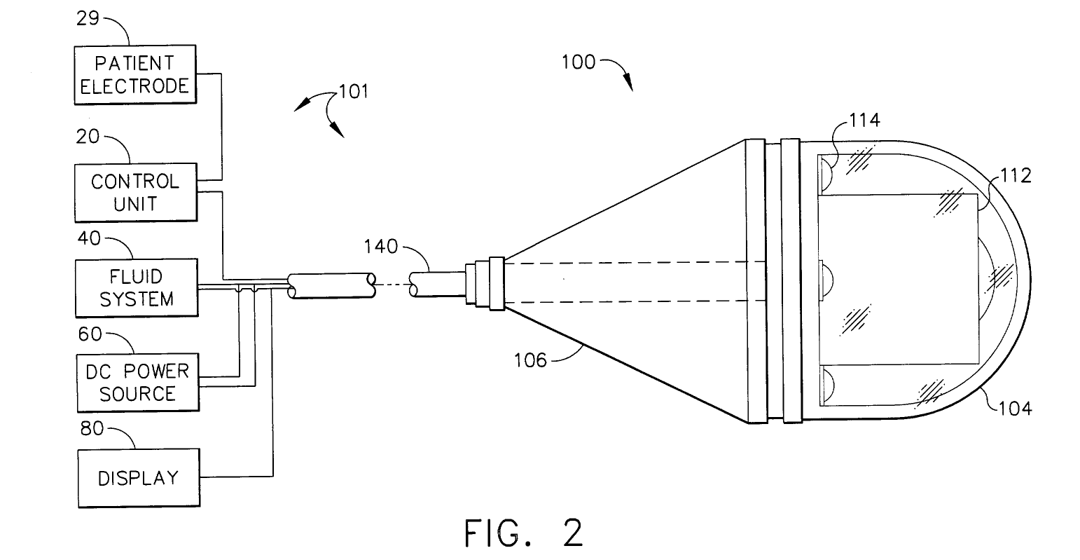

FIG. 2 shows a medical device 101, which generally comprises a capsule 100, an umbilicus 140, a control unit 20, a DC power source 60, a display 80, a patient electrode 29, and a fluid system 40. Capsule 100, for this embodiment, has a semi-spherical, leading end 104, a conical, trailing end 106, and is sized to slide easily through the anus of the patient. In general, the outside of capsule 100 is smooth and streamlined for sliding easily through the colon. Trailing end 106 of capsule 100 is conical so that when the colon constricts due to electrical stimulation, capsule 100 moves in a forward direction with attached umbilicus 140 trailing behind. Many other suitable shapes for capsule 100 are possible. Umbilicus 140 is flexible and is approximately as long as the flexible shaft of a colonoscope, which typically has a length of about 1.7 meters. Umbilicus 140 is preferably made from a thinwall, flexible plastic or rubber tube suitable for transporting fluid between fluid system 40 and capsule 100.

Capsule 100 can be constructed from one or more of numerous materials that are rigid relative to the soft tissue of the body. These materials include metals, elastomers, and plastics. Preferably, capsule 40 is made from injection molded plastic in two or more pieces that are assembled with the other components. Suitable plastics include polycarbonate, polyetherimide, and polyethylene.

In FIG. 2, control unit 20 is shown electrically connected to capsule 100 to provide electrical pulses of a first polarity and of a desired frequency. Patient electrode 29 is attached to an external surface of the patient, and receives electrical pulses of a second polarity from control unit 20, wherein the second polarity is opposite of the first polarity. Control unit 20 comprises a frequency generator that provides at least one electrical waveform. Suitable waveforms include sinusoidal waves, square waves, triangular waves, and combinations. Control unit 20 also includes a constant current source, such as the STIMULUS ISOLATOR commercially available from World Precision Instruments of Sarasota, Fla. Control unit 20 allows the operator to activate and deactivate electrical stimulation to the colon, thus controlling the intraluminal propulsion of capsule 100. Control unit 20 also allows the operator to control the electrical pulse frequency of the stimulation, which may be generally uniform or varying. A suitable pulse frequency is approximately in the range of 5 to 20 Hz, but can be as high as approximately 1000 Hz. Control unit 20 also allows the operator to control electrical stimulation current amplitude. A suitable electrical stimulation current amplitude is approximately in the range of 10 to 50-mA, but can be as high as about 100-mA. However, it is also possible for waveform, frequency, and current amplitude to operate according to predetermined values set in control unit 20, therefore not requiring operator adjustment during the medical procedure. One particularly suitable electrical stimulation type is a half duty cycle, 15 Hz, 30-mA square wave.

DC power source 60 provides electrical power to a lighting device 114 and a visualization device 112, both contained in leading end 104 of capsule 100. Lighting device 114 may be a plurality of white light emitting diodes (LED's) which are commercially available from Nichia (www.nichia.com) model number NSPWF50BS. Lighting device 114 also may be, for example, an incandescent lamp. Visualization device 112 may be a complementary metallic oxide semiconductor (CMOS) camera, which is commercially available from Omnivision Technologies, Inc. (www.ovt.com) as Model Number OV7910. Visualization device 112 may also be a charged couple device (CCD) camera. Display 80 comprises a monitor having a video format (NTSC, PAL) required by the visualization device for visually displaying the image transmitted by visualization device 112.

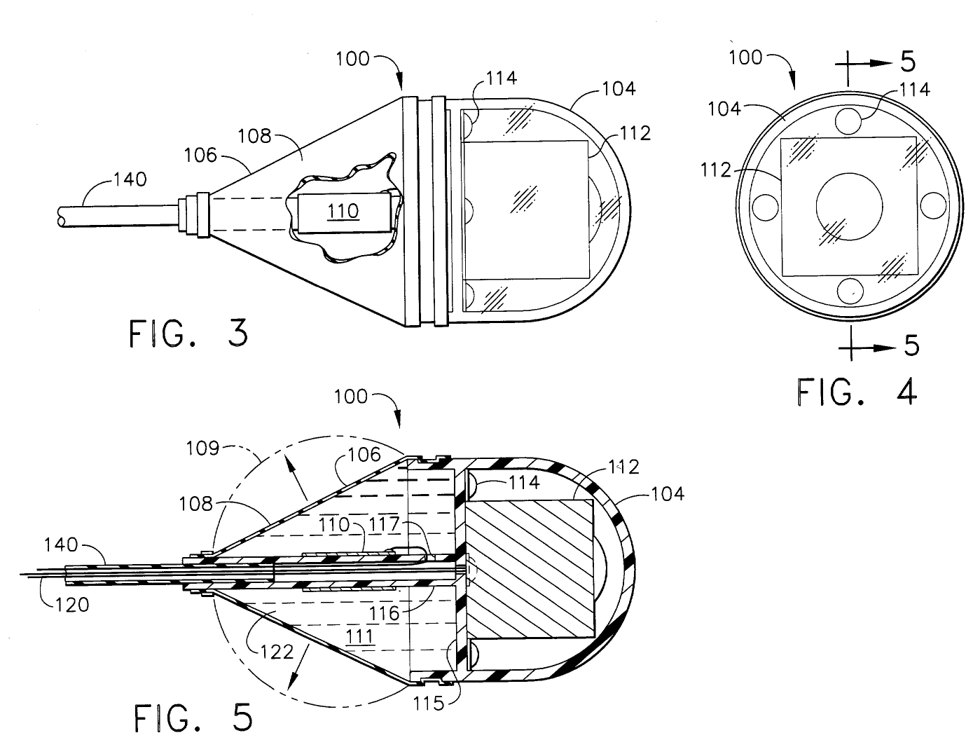

FIGS. 3 and 4 show a side and proximal end view, respectively, of capsule 100. Leading end 104 is hollow and can be made from a transparent material such as injection molded polycarbonate. Visualization device 112 is mounted inside of leading end 104 and surrounded by lighting devices 114. Trailing end 106 comprises a selectively expandable member, such as a balloon 108 made from an elastomeric material such as latex rubber or silicone rubber. Balloon 108 has a thickness approximately in the range of 0.08 to 0.40 mm. In FIG. 3, a portion of balloon 108 is removed to reveal a first electrode 110, which is electrically connected to control unit 20 (FIG. 2). In this embodiment, balloon 108 has a generally conical shape when in a non-inflated configuration.

FIG. 5 is a sectional view of capsule 100. Visualization device 112 and lighting devices 114 are mounted to a partition 115, which divides leading end 104 and trailing end 106. A conduit 116 extends longitudinally through the center of trailing end 106. Conduit 116 provides a channel from umbilicus 140 to the inside of leading end 104 for a plurality of insulated wires 120 that electrically service visualization device 112, lighting device 114, and electrode 110. In this embodiment, electrode 110 is a metallic tube fitting closely around conduit 116. The space around conduit 116 and inside of balloon 108 defines a fluid chamber 111, shown filled with a fluid 122. Fluid 122 is preferably saline, but can also comprise one or more different electrically conductive solutions. Fluid system 40 (FIG. 2) supplies and pressurizes fluid 122 to fluid chamber 111 through a port 117 in conduit 116 according to control unit 20 commands or by operator control. When fluid 122 is pressurized, balloon 108 changes to an inflated configuration as indicated by phantom lines 109 in FIG. 5. When in the inflated configuration, balloon 108 comes into intimate contact with the stretched colon. The increased volume of fluid 122 inside of balloon 108 provides an effective capacitive electrical pathway between electrode 110 having a first electrical polarity and the colon, which is in electrical communication with patient electrode 129 having a second (opposite) electrical polarity. While balloon 108 is in the inflated configuration, control unit 20 electrically stimulates the contractile tissue in the colon wall. The colon contraction against balloon 108 propels capsule 100 in a forward direction. Balloon 108, when filled with fluid 122, presents a soft and spring-like surface to the delicate inside of the contracting colon.

Balloon 108 can be continuously inflated during the entire time control unit 20 continuously stimulates the colon until capsule 100 traverses the desired length of colon. Balloon 108 may also be inflated then deflated periodically at a rate, for example, equal to the rate of electrical stimulation. For example, balloon 108 may be inflated for one second while control unit 20 electrically stimulates the colon. During the subsequent second, balloon 108 may be deflated, while electrical stimulation is deactivated. This is repeated until capsule 100 traverses the desired length of colon. Other inflation/stimulation cycles are possible.

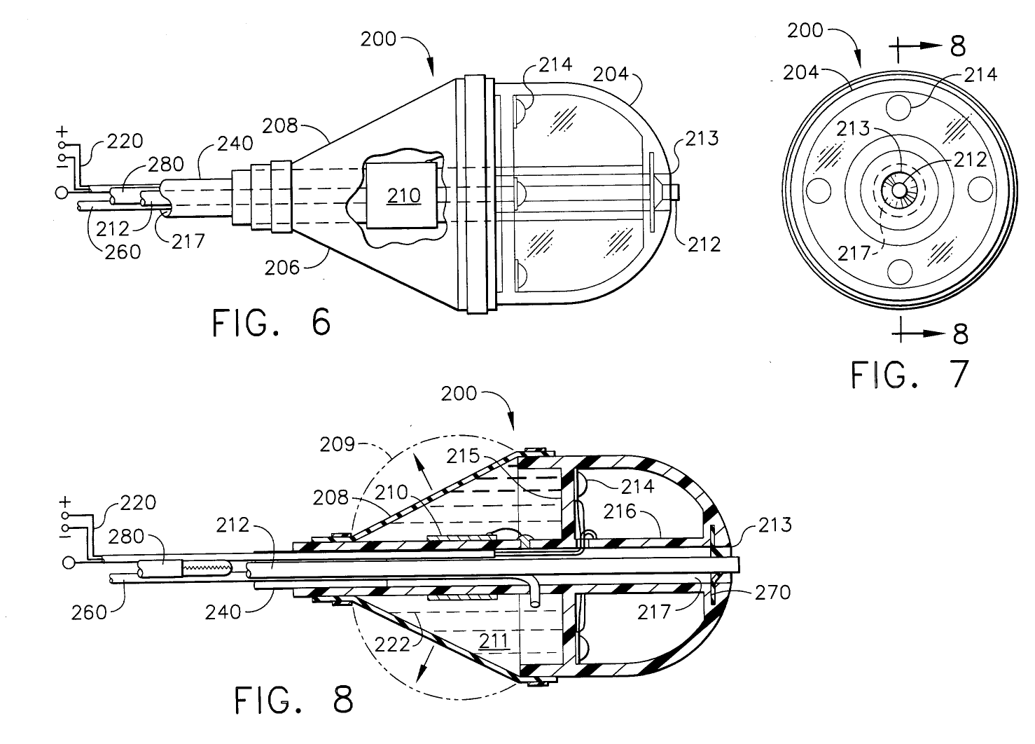

FIG. 6, FIG. 7, and FIG. 8 show another embodiment of a self-propelled, intraluminal device. A capsule 200 and an umbilicus 240 include a working channel 217 for providing access with a medical instrument 280 and a fiber optic bundle 212 from outside the colon to the inside. Capsule 200 includes a leading end 204, a trailing end 206, and a conduit 216 extending longitudinally therethrough. Conduit 216 comprises the distal portion of working channel 217. Leading end 204 retains a seal 270 that closes a port 213 on the distal end of working channel 217. Seal 270 is preferably made of a thin silicone membrane with a tiny central hole that stretches to allow passage of medical instrument 280 or fiber optic bundle 212. Leading end 204 is preferably injection molded from a clear, rigid plastic such as polycarbonate. Lighting device 214, which in this embodiment is shown as a plurality of white LED's, is mounted on partition 215 to illuminate the bodily lumen immediately distal to capsule 200. Lighting device 214 is electrically connected to DC power source 60 as for the previous embodiment shown in FIG. 2. Fiber optic bundle 212 passes through working channel 217 to an external camera and display, or may be attached to an optical eyepiece (not shown) for direct viewing by the operator. Trailing end 206 comprises a balloon 208 having a generally conical shape when in a deflated configuration. Balloon 208 has an inflated configuration indicated by phantom lines 209 when fluid 222 is pressurized. A fluid tube 260 passes through working channel 217 to fluidly connect a fluid chamber 211 to fluid system 40 (FIG. 2). A first electrode 210 can be in the form of a metal cylinder mounted over conduit 216 inside of fluid chamber 211. First electrode 210 is electrically connected to control unit 20 (FIG. 2) and has a first electrical polarity. A patient electrode 29 (FIG. 2) having a second (opposite) polarity electrically connects to an external surface of the patient. First electrode 210 capacitively connects to the colon, as was described for the previous embodiment.

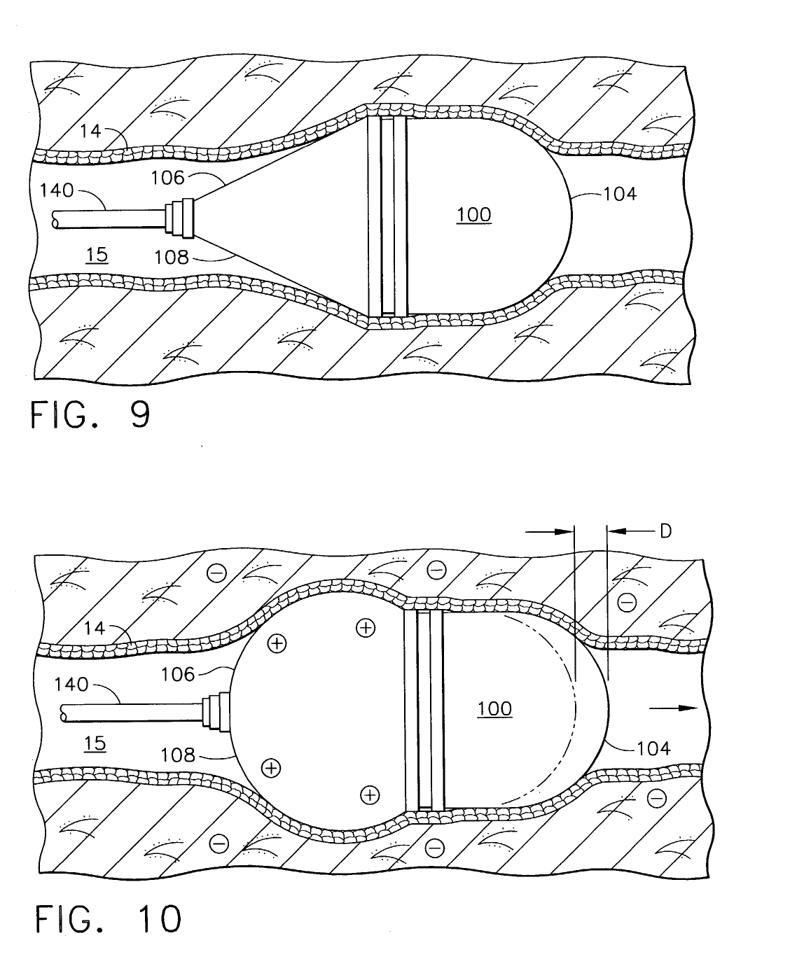

FIG. 9 and FIG. 10 show capsule 100 and umbilicus 140 traversing through bodily lumen 15 of the colon. (Capsule 200 traverses through the colon in a similar manner.) In FIG. 9, capsule 100 is in a deflated configuration and electrical stimulation is deactivated. Balloon 108 of trailing end 106 has a conical shape and is significantly separated from wall 14 of the colon. In FIG. 10, balloon 108 is in the inflated configuration and is intimately contacting wall 14 of the colon. Electrical stimulation is activated as indicated by the positive polarity of balloon 108, and the opposing negative polarity of wall 14. Leading end 104 has moved a distance D in the forward (right) direction. Depending on the strength of contraction of the colon and the pressure of fluid 122 inside of balloon 108, balloon 108 may compress to an intermediate shape that is neither an inflated shape as shown in FIG. 10, or a deflated shape as shown in FIG. 9. Balloon 108, therefore, exerts a spring force against wall 14 of the colon as the colon contracts, thus aiding capsule 100 to move in the forward direction.

The medical devices shown in FIGS. 2-10 generally have the same method of use, which can comprise the following steps. The medical device is provided and the operator attaches the patient electrode to an external surface of the patient. The operator inserts the capsule and a portion of the umbilicus into a bodily lumen while the capsule is in the contracted configuration. The operator changes the capsule to an expanded configuration. The operator activates the control unit to capacitively couple the first electrode through the balloon to the wall of the bodily lumen to electrically stimulate the wall of the bodily lumen. The operator monitors the movement of the umbilicus into the bodily lumen. The operator deactivates the control unit to stop the electrical stimulation. The operator changes the capsule to the contracted configuration. The sequence can be repeated, as desired, to move the capsule to a desired position within the lumen (e.g. within the gastro-intestinal tract) The operator can pull the umbilicus and remove the capsule from the body upon completion of the procedure being performed.

The method of use of the medical devices shown may also include the step of directing a medical instrument from a point outside the lumen, through a working channel extending through the umbilicus and into the capsule, such as to access tissue, and/or remove tissue from inside of the bodily lumen. The method of use may also including providing a medical device that includes a visualization device electrically connected to an electrical power source and a display, and a lighting device electrically connected to an electrical power source, and for using the medical device to visualize inside the bodily lumen.

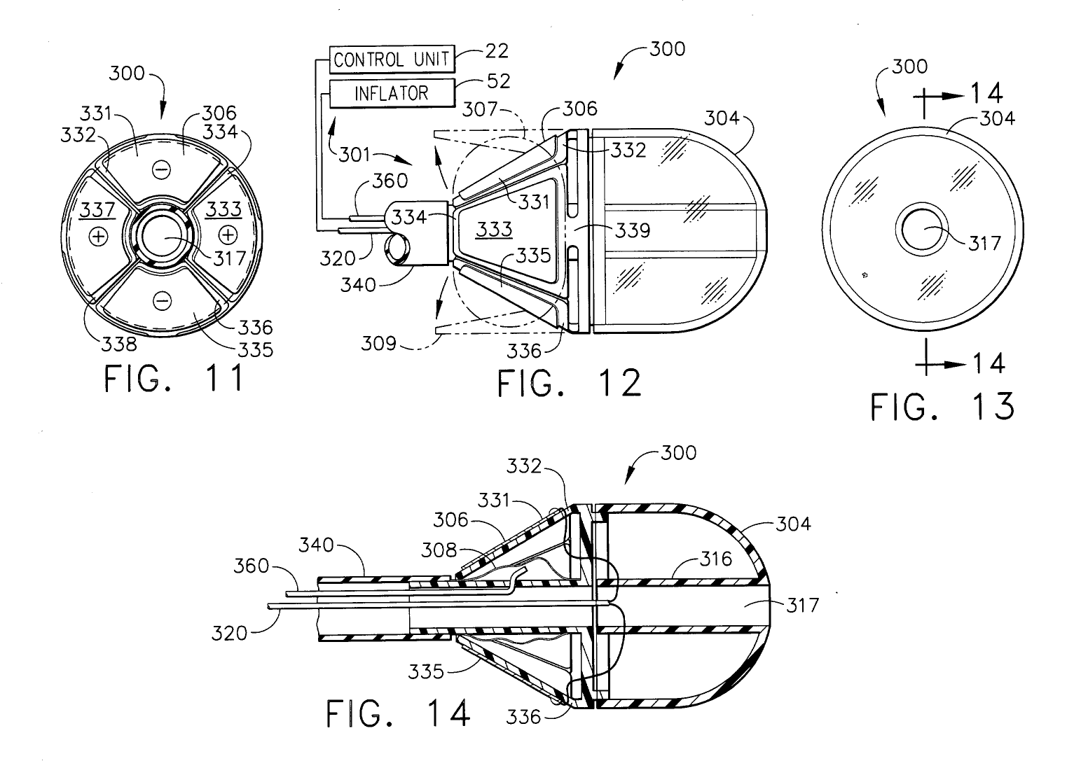

FIG. 12 shows a medical device 301 that comprises a capsule 300, an umbilicus 340, a control unit 22, and an inflator 52. Capsule 300 comprises a leading end 304 and a trailing end 306. Referring also to FIG. 11 and FIG. 13, capsule 300 further comprises a first wing 332 with a first movable electrode 331, a second wing 334 with a second movable electrode 333, a third wing 336 with a third movable electrode 335, and a fourth wing 338 with a fourth movable electrode 337. First, second, third, and fourth wings, 332, 334, 336, and 338, respectively, are normally in a contracted position as shown in FIG. 12, so that the operator may pull umbilicus 340 and move capsule 300 in a reverse direction, but are movable to an expanded position, which is indicated by phantom lines 309, for more intimate contact with the colon and improved electrical stimulation of contractile tissue in the colon while capsule 300 moves in a forward direction. First electrode 331 and third electrode 335 have a first electrical polarity as indicated by negative signs in FIG. 11. Second electrode 333 and fourth electrode 337 have a second electrical polarity opposite of the first electrical polarity, as indicated by positive signs in FIG. 11. It is possible for capsule 300 to have only one movable electrode of a first polarity if a patient electrode of a second polarity is attached to an external surface of the patient. It is also possible to have two, three, or more than four movable electrodes having first and second polarities, preferably on alternating electrodes. It is also possible to have more than one movable electrode of a first polarity if used with a patient electrode of a second polarity.

Actuation of the wings, such as by inflation of the ballon, causes electrodes 331-337 to move with respect to each other. Relative movement of the electrodes with respect to each other and to the main portion of the capsule allows for positioning of the electrodes with respect to the tissue that would not generally be practical with electrodes fixed to a single surface of a capsule.

FIG. 14 is a sectional view of capsule 300 and the distal portion of umbilicus 340. A conduit 316 extends longitudinally through the center of leading end 304 and trailing end 306, and contains a working channel 317. A plurality of wires 320 pass through working channel 317 to capsule 300 and electrically connect first, second, third, and fourth movable electrodes, 331, 333, 335, 337 respectively, to control unit 22. A tube 360 fluidly connects a balloon 308 (shown in a deflated configuration) to inflator 52. A fluid such as air, saline, or water, may be injected into balloon 308 by inflator 52 according to commands from control unit 22, or optionally by manual control, to change balloon 308 to an inflated configuration as indicated by phantom lines 307, thus forcing first, second, third, and fourth wings, 332, 334, 336, and 338, to move to the expanded configuration, as indicated by phantom lines 309. Working channel 317 may be used for access from outside the body to inside the colon with a medical instrument, a small diameter fiber optic bundle device for illumination and visualization, or for the application or removal of fluids.

Still referring to FIG. 14, leading end 304 and conduit 316 may be injection molded as one piece from a rigid and bendable plastic such as polycarbonate. Trailing end 306 and the proximal end of conduit 316 are preferably injection molded as one piece, and with first, second, third, and fourth wings, 332, 334, 336, and 338, in the normally closed position, also from a plastic such as polycarbonate. Each of first, second, third, and fourth wings, 332, 334, 336, and 338, have a plastic hinge such as hinge 339 on third wing 334 shown in FIG. 12. Hinge 339 flexes when balloon 308 is inflated, and provides resilience to return wing 334 to a contracted configuration when balloon 308 is deflated. Other spring types and configurations can be used to bias the wings to a desired configuration, such as a contracted configuration.

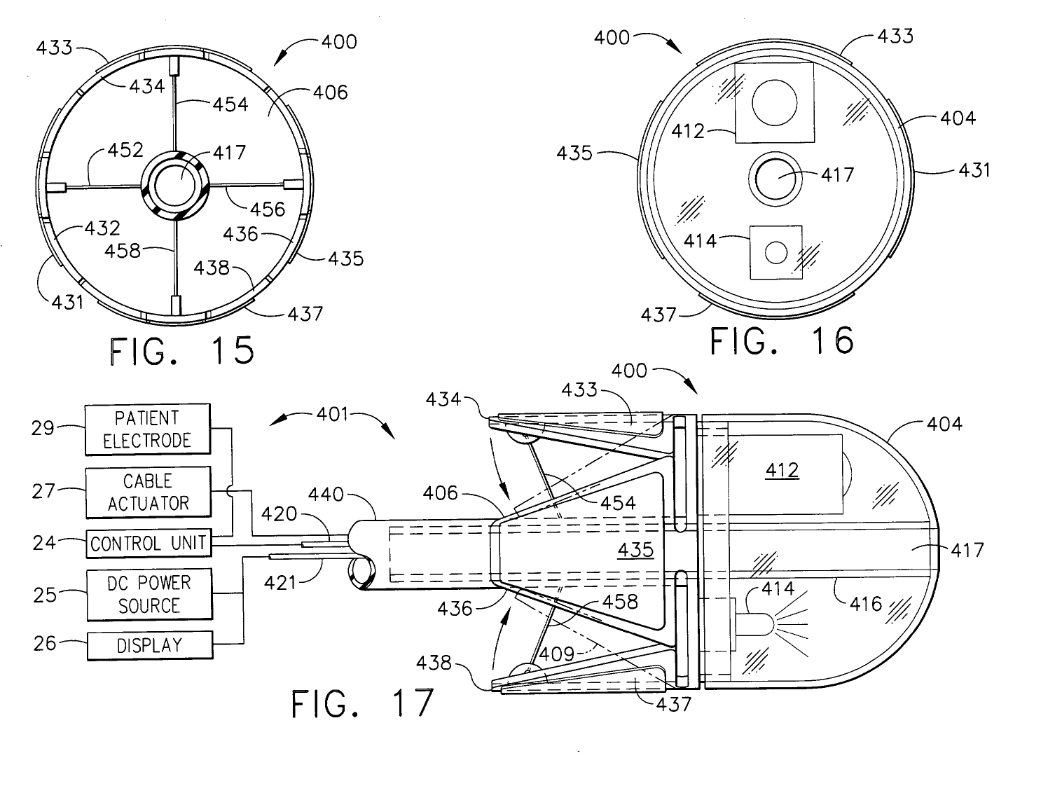

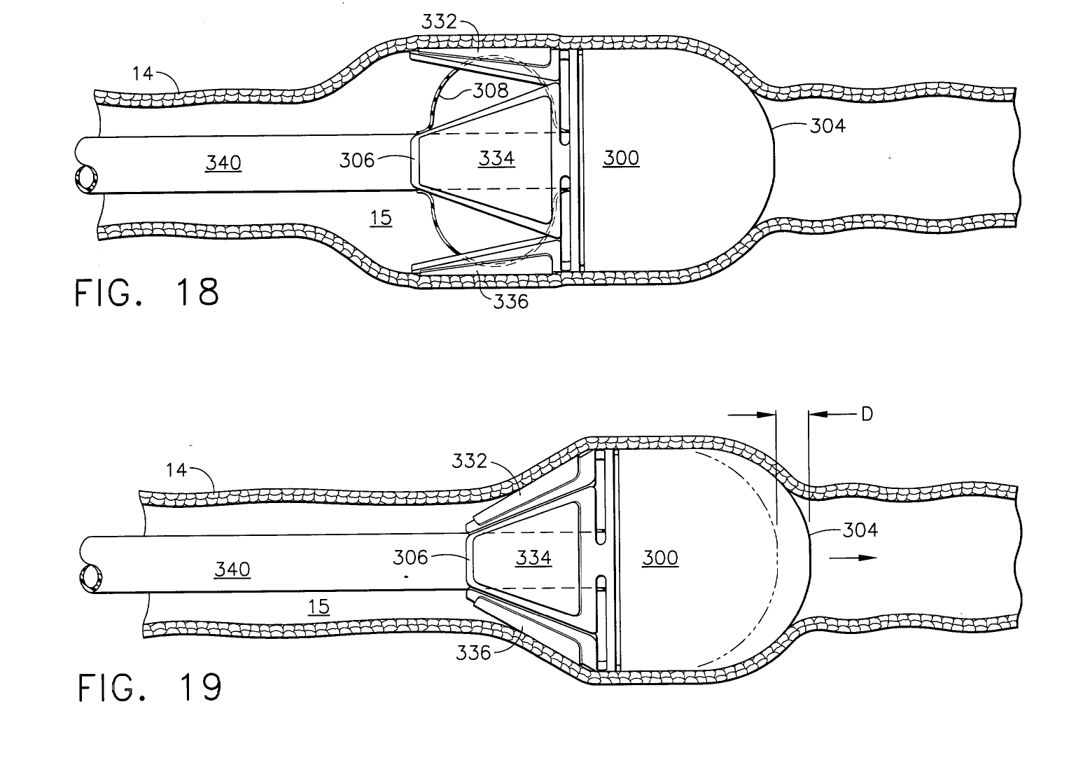

FIG. 15, FIG. 16, and FIG. 17 show a medical device 401 that comprises a capsule 400, an umbilicus 440, a patient electrode 29, a cable actuator 27, a control unit 24, a DC power source 25, and a display 26. Capsule 400 includes a leading end 404, which is hollow and made from a transparent material such as polycarbonate. Leading end 404 contains a visualization device 412, which may be a CMOS or CCD camera as described for medical device 100 in FIG. 5. Leading end 404 also contains a lighting device 414, also as described for medical device 100. Visualization device 412 and lighting device 414 are electrically connected to DC power source 25 and display 26 by wiring 421. Capsule 400 further includes a trailing end 406 having a first wing 432, a second wing 434, a third wing 436, and a fourth wing 436, each of which are movable between an expanded configuration as shown in FIG. 17, or in a contracted position as indicated by phantom lines 409 in FIG. 17. First wing 434 has a first electrode 431, second wing 434 has a second electrode 433, third wing 436 has a third electrode 435, and fourth wing 438 has a fourth electrode 437. As for medical device 300 of FIG. 14, electrodes 431, 433, 435, and 437 may have alternating electrical polarities (in which case patient electrode 29 would not be used) or may have a first electrical polarity and used in combination with patient electrode 29 having a second (opposite) polarity.

Trailing end 406 is preferably injection molded from a bendable plastic such as polycarbonate so that first, second, third, and fourth wings, 432, 434, 436, and 438 are normally in the expanded position as shown in FIG. 17. First wing 432 attaches to a first cable 452, second wing 434 attaches to a second cable 454, third wing 436 attaches to a third cable 456, and fourth wing 438 attaches to a fourth cable 458. First, second, third, and fourth cables, 452, 454, 456, and 458, respectively, extend through umbilicus 440 and operably connect to cable actuator 27, enabling the operator to apply or release cable tension, thus changing capsule 400 between a contracted configuration for movement in the reverse direction in the colon and an expanded configuration for improved electrical contact with the colon during movement in the forward direction. In yet another embodiment, cables 452-458 can be replaced by other suitable mechanical mechanisms, such as linkage (not shown) or other suitable mechanism to actuate the wings 432-438 between the extended and contracted configurations. In such an embodiment, a non-biasing hinge can be used to connect wings to the body of the capsule, and the linkage (such as an assembly of one or more hinged or telescoping links) or other suitable mechanism can be used to position the wings in the desired configuration.

Still referring to FIG. 17, capsule 400 includes a conduit 416 extending longitudinally through leading end 404 and trailing end 406. A working channel 417 extends through conduit 416 and umbilicus 440, allowing access with medical instruments from outside the body to inside the colon, or transfer of fluids into and out of the colon.

FIG. 18 and FIG. 19 depict capsule 300 of FIG. 112 traversing bodily lumen 15 of the colon. Capsule 400 of FIG. 17 would traverse bodily lumen 15 in a similar manner. In FIG. 18, trailing end 306 of capsule 300 is in an expanded configuration due to the inflation of balloon 308, thus bringing first, second, third, and fourth electrodes, 332, 334, 336, and 338, respectively, into intimate contact with wall 14 of the colon, and optimizing electrical stimulation of contractile tissue in wall 14. In FIG. 19, the electrical stimulation and subsequent contraction of wall 14 of the colon forces trailing end 306 to change to the contracted configuration, propelling capsule 300 and the trailing umbilicus 340 in the forward direction a distance D. When control unit 24 (FIG. 17) deactivates electrical stimulation, trailing end 306 resumes the expanded configuration as shown in FIG. 18 due to the fluid pressure inside of balloon 308. To remove capsule 300 from the colon, the operator deflates balloon 308 so that trailing end 306 changes to the contracted configuration, and the operator may then pull umbilicus 340 gently to move capsule 300 in the reverse direction.

Medical devices 301 and 401 generally have the same method of use, which can comprise the following steps. The medical device is provided to the operator, and the operator inserts the capsule and a portion of the umbilicus into a bodily lumen while the capsule is in a contracted configuration. The operator changes the capsule to an expanded configuration. The operator activates the control unit to electrically stimulate the wall of the bodily lumen. The operator monitors the movement of the umbilicus into the bodily lumen. The operator deactivates the control unit to stop the electrical stimulation. The operator changes the capsule to the contracted configuration. The sequence can be repeated, as desired. The operator can pull the umbilicus and remove the capsule from the bodily lumen when the procedure being performed is completed.

The method of use of medical device 301 and 401 may also include the step of providing a medical device that includes a working channel, and of accessing the inside of the bodily lumen with a medical instrument from outside the bodily lumen. The method of use may also including providing a medical device that includes a visualization device electrically connected to an electrical power source and a display, and a lighting device electrically connected to an electrical power source, and for using the medical device to visualize inside the bodily lumen.

In an alternative embodiment, it may be desirable to control movement of the wings individually, so that each electrode can be individually positioned as desired. In the embodiments described above, the lumen tissue stimulating device employees an electrical stimulus to facilitate travel of the capsule 40 through the lumen. In other embodiments, other stimuli may be used, including without limitation, sonic energy (such as ultrasonic energy), light energy, or chemical stimuli (such as by controlled deposition of a liquid from the capsule to the lumen wall to cause contraction of the lumen wall).

It will be recognized that equivalent structures may be substituted for the structures illustrated and described herein and that the described embodiment of the invention is not the only structure which may be employed to implement the claimed invention. In addition, it should be understood that every structure described above has a function and such structure can be referred to as a means for performing that function.

While numerous embodiments of the present invention have been disclosed, it will be obvious to those skilled in the art that such embodiments are provided by way of example only. Numerous variations, changes, and substitutions will now occur to those skilled in the art without departing from the invention. Accordingly, it is intended that the invention be limited only by the spirit and scope of the appended claims.

What is claimed is:

1. A medical device comprising:
a capsule adapted for travel in a body lumen, wherein the capsule comprises a movable portion, the movable portion movable from a contracted configuration to an expanded configuration;
a lumen tissue stimulator associated with the movable portion of the capsule.

2. The medical device of claim 1 wherein the movable portion comprises a plurality of wings.

3. The medical device of claim 1 wherein the tissue stimulator comprises at least one electrode.

4. The medical device of claim 1 wherein the movable portion is biased to a selected one of a contracted configuration and an expanded configuration.

5. The medical device of claim 1 wherein the movable portion is biased to a contracted configuration.

6. The medical device of claim 1 wherein the movable portion comprises a plurality of wings, and wherein an electrode is associated with each wing.

7. The medical device of claim 1 comprising an actuator for positioning the movable portion in the expanded configuration.

8. The medical device of claim 7 wherein the actuator comprises an inflatable member.

9. The medical device of claim 7 wherein the actuator comprises a mechanism.

10. The medical device of claim 7 wherein the actuator comprises a cable.

11. A medical device comprising:
a capsule for introduction into a body lumen, said capsule comprising an inflatable member comprising a conductive fluid; and
at least one electrode associated with the inflatable member.

12. The medical device of claim 11 further comprising an umbilicus.

13. The medical device of claim 11 further comprising a control unit for controlling movement of the capsule through the body lumen.

14. The medical device of claim 11 comprising a visualization device and a lighting device for visualizing inside of the body lumen.

15. The medical device of claim 14 wherein said visualization device is a CMOS camera.

16. The medical device of claim 14 wherein said visualization device is a CCD camera.

17. The medical device of claim 11 further comprising a fluid system for supplying conductive fluid to the inflatable member.

18. The medical device of claim 17 wherein said fluid system is adapted to pressurize and depressurize the conductive fluid periodically.

19. The medical device of claim 18 wherein said fluid system is adapted to pressurizes and depressurize the conductive fluid in association with 30 application of electrical pulses to an electrode.

20. The medical device of claim 12 further comprising a working channel extending through said umbilicus and said capsule, and providing access with a medical instrument from outside the body lumen to the inside of the bodily lumen.

21. A method of accessing the inside of a body lumen, said method comprising:
providing a capsule, the capsule supporting at least one electrode; and
actuating a portion of the capsule to move the electrode into a desired position with respect to tissue within the lumen.

22. The method of claim 21 wherein the step of actuating comprises inflating a portion of the capsule.

23. The method of claim 21 wherein the step of actuating comprises actuating a cable.

24. The method of claim 21 wherein the step of actuating a portion of the capsule provides an expanded configuration of the capsule.

25. The method of claim 21 wherein the step of actuating a portion of the capsule comprises movement of a plurality of electrodes with respect to each other.
